(19) United States
(12) Patent Application Publication
(10) Pub. No.: US 2024/0358297 A1
(43) Pub. Date: Oct. 31, 2024

(54) Blood Sample Collection System with Integrated Scrubbing Cap and Method of Use Thereof

(71) Applicant: Becton, Dickinson and Company, Franklin Lakes, NJ (US)

(72) Inventor: Jonathan Karl Burkholz, Salt Lake City, UT (US)

(21) Appl. No.: 18/643,561

(22) Filed: Apr. 23, 2024

Related U.S. Application Data
(60) Provisional application No. 63/461,712, filed on Apr. 25, 2023.

(51) Int. Cl. A61B 5/15

(52) U.S. Cl. CPC A61B 5/150328; A61B 5/15003; A61B 5/150053

(57) ABSTRACT

Provided herein is a system including a container assembly having a cap and defining a reservoir, and a fluid access assembly having a housing defining an interior, a fluid access component, and a fluid connector component, the housing having a distal end and a proximal end, the fluid access component extending from the housing distal end into the interior of the housing, the fluid access component defining a lumen, the fluid connector component disposed on the distal end of the housing. A scrubbing cap is provided at or adjacent a distal end of the fluid access assembly, with the scrubbing cap having a scrubbing insert and a housing defining a cavity configured to retain the scrubbing insert therein.

### CROSS-REFERENCE TO RELATED APPLICATION

The present application claims priority to U.S. Provisional Application No. 63/461,712 entitled “Blood Sample Collection System with Integrated Scrubbing Cap and Method of Use Thereof” filed Apr. 25, 2023, the entire disclosure of which is hereby incorporated by reference.

### BACKGROUND OF THE INVENTION

### Field of the Invention

The present disclosure relates to a blood sample collection system with an integrated scrubbing cap and a method of use thereof.

### Description of Related Art

Catheters are commonly used to administer fluids into and draw fluids (i.e., blood) out of the body, with patients in a variety of settings, including in hospitals and in home care, being administered such fluids or having blood drawn via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

In some applications, a VAD is used for collection of a blood specimen in connection with performing of a blood culture. Blood cultures are often used as a tool to detect the presence of bacteria or fungi in a blood sample of a patient, to identify the type of bacteria or fungi present, and to direct the treatment of the patient. The current practice of collecting a venous blood sample for blood culture testing involves a venipuncture with a blood collection set with an attached luer-lock access device (LLAD). During the insertion process, the venipuncture device may pick up microbes from the skin and insertion process and later transfer such microbes into the blood culture sample leading to a false positive test result for sepsis. To minimize the risk of a false positive, a discard sample is typically taken to remove the initial volume of blood that more likely contains microbes from the skin, such as discarding the first 1-10 ml of blood drawn. However, this process also introduces some risk, as the discard sample collection introduces additional non-sterile steps, techniques, and connections that increase the risk of introducing microbes into the subsequent blood culture sample.

In some prior art devices, such as that described in US Publication 2021/0196167 to Velano Vascular, Inc., the above complications are addressed by adding an adapter to the LLAD that holds the discard sample in a pre-advanced position, allowing the entire assembly (including the discard sample) to be sterilized, so as to thereby reduce the number of non-sterile steps and connections during the blood culture sample collection procedure. This reduces the overall risk of introducing microbes into the blood culture sample and therefore reduces the risk of a false positive.

Although a LLAD as described above addresses some of the risk of a false positive blood culture sample, it does not address the risk of a clinician not disinfecting or ineffectively disinfecting an access port or connector of the VAD prior to connecting the LLAD thereto. That is, clinical practitioners often use alcohol wipes for scrubbing and disinfecting the access port before connecting a LLAD thereto, but use of alcohol wipes for scrubbing and disinfecting the access port is oftentimes not completely effective in disinfecting all internal/external surfaces of the access port, including (potentially) the intricate thread areas around the a luer portion thereof that couples with the LLAD. Additionally, it is possible in some instances for the clinician to forget to wipe down the access port prior to connection of the LLAD. If the disinfection of the access port is not performed properly, a false positive blood culture sample may arise.

Accordingly, a need exists in the art for a means for ensuring that a clinician effectively disinfects the access port of the VAD prior to attaching a blood collection device thereto.

### SUMMARY OF THE INVENTION

Provided herein is a system including a container assembly having a cap and defining a reservoir, with the container assembly having a distal end and a proximal end with the cap disposed at the distal end of the container assembly. The system also includes a fluid access assembly having a housing defining an interior, a fluid access component, and a fluid connector component, the housing having a distal end and a proximal end, the fluid access component extending from the distal end of the housing into the interior of the housing, the fluid access component defining a lumen, the fluid connector component disposed on the distal end of the housing. The system further includes a scrubbing cap provided at or adjacent a distal end of the fluid access assembly, with the scrubbing cap having a scrubbing insert and a housing defining a cavity configured to retain the scrubbing insert therein.

In some embodiments, the fluid connector component comprises a luer connector configured to be coupled to an access port of a vascular access device, and wherein a connector portion of the scrubbing cap is engaged with the luer connector to secure the scrubbing cap to the fluid access assembly.

In some embodiments, the luer connector comprises one of a slip luer, a threaded luer, or a luer lock with collar.

In some embodiments, the system includes an adapter including a first engagement feature and a second engagement feature, the first engagement feature of the adapter configured to releasably engage with the cap of the container assembly and the second engagement feature of the adapter configured to releasably engage with a housing engagement feature of the fluid access assembly such that, in a first configuration in which the first engagement feature of the adapter is engaged with the cap and the second engagement feature of the adapter is engaged with the engagement feature of the fluid access assembly, the cap of the container assembly is at least partially disposed within the interior of the housing and spaced from the fluid access component.

In some embodiments, the container assembly is configured to be transitioned from the first configuration to a second configuration via translating the container assembly toward the first end of the fluid access assembly such that the cap is disengaged from the first engagement feature of the adapter and the fluid access component pierces a rescalable membrane of the cap such that the reservoir of the container assembly is in fluidic communication with the fluid connector component and the connection portion via the lumen of the fluid access component.

In some embodiments, the system includes an extension member having a distal end and a proximal end, the proximal end of the extension member coupled to the fluid connector component, wherein the extension member is configured to reduce hemolysis of a blood sample passing therethrough, and wherein a connector portion of the scrubbing cap is engaged with the distal end of the extension member to secure the scrubbing cap to the extension member.

In some embodiments, the extension member includes a connector interface disposed at the distal end and configured to couple the extension member to an access port of a vascular access device and a flexible tube fluidly coupled to the connector interface and configured to be coupled to the fluid connector component of the fluid access assembly, wherein the flexible tube is configured to reduce hemolysis of a blood sample passing therethrough.

In some embodiments, the extension member includes a compact connector having a proximal connector portion configured to couple the compact connector to the fluid connector component, a distal connector portion configured to couple the compact connector to an access port of a vascular access device and a central portion shaped and configured to reduce hemolysis of a blood sample passing therethrough.

In some embodiments, the connector interface or the distal connector comprises one of a threaded luer, a slip luer, a threaded luer lock with collar, a blunt plastic cannula, a male luer, a cannula for PRN access, a needle-free connector, or a needle access cannula.

In some embodiments, the connector portion of the scrubbing cap secures the scrubbing cap via a twist-type engagement, and wherein the scrubbing cap is removable to enable connection of the system to an access port of a vascular access device.

In some embodiments, the scrubbing insert comprises a resilient material including an antimicrobial solution or agent absorbed therein configured to disinfect a surface of the access port of the vascular access device.

In some embodiments, the scrubbing cap includes a seal attached over the cavity to seal the scrubbing insert within the cavity.

Also provided herein is a method of using a blood sample collection system. The method includes providing a blood sample collection system comprising a container assembly having a cap and defining a reservoir, the container assembly having a distal end and a proximal end, the cap disposed at the distal end of the container assembly, a fluid access assembly comprising a housing defining an interior, a fluid access component, and a fluid connector component, the housing having a distal end and a proximal end, the fluid access component extending from the distal end of the housing into the interior of the housing, the fluid access component defining a lumen, the fluid connector component disposed on the distal end of the housing, and a scrubbing cap provided on or adjacent the distal end of the housing, the scrubbing cap comprising a scrubbing insert and a housing defining a cavity configured to retain the scrubbing insert therein. The method also includes positioning the scrubbing cap over an access port of a vascular access device, such that the access port contacts the scrubbing insert, and cleaning the access port with the scrubbing insert.

In some embodiments, the fluid connector component comprises a luer connector configured to be coupled to the access port, and wherein a connector portion of the scrubbing cap is engaged with the luer connector to secure the scrubbing cap to the fluid access assembly. In some embodiments, the method also includes detaching the scrubbing cap from the luer connector subsequent to cleaning of the access port and coupling the luer connector to the access port, to place the fluid access assembly in fluid connection with the vascular access device.

In some embodiments, the system comprises an extension member having a distal end and a proximal end, the extension member configured to reduce hemolysis of a blood sample passing therethrough, and wherein a connector portion of the scrubbing cap is engaged with the distal end of the extension member to secure the scrubbing cap to the extension member.

In some embodiments, the method also includes detaching the scrubbing cap from the distal end of the extension member subsequent to cleaning of the access port and coupling the distal end of the extension member to the access port, to place the fluid access assembly in fluid connection with the vascular access device.

In some embodiments, the system comprises an adapter including a first engagement feature and a second engagement feature, the first engagement feature of the adapter configured to releasably engage with the cap of the container assembly and the second engagement feature of the adapter configured to releasably engage with a housing engagement feature of the fluid access assembly such that, in a first configuration in which the first engagement feature of the adapter is engaged with the cap and the second engagement feature of the adapter is engaged with the engagement feature of the fluid access assembly, the cap of the container assembly is at least partially disposed within the interior of the housing and spaced from the fluid access component.

In some embodiments, the method also includes translating the container assembly toward the first end of the fluid access assembly and relative to the adapter such that the cap is disengaged from the first engagement feature of the adapter and the fluid access component pierces a resealable membrane of the cap such that the reservoir of the container assembly is in fluidic communication with the fluid connector component via the lumen of the fluid access component, decoupling the second engagement feature of the adapter from the engagement feature of the fluid access assembly, and translating the container assembly away from the first end of the fluid access assembly and out of the interior of the fluid access assembly such that the container assembly and the adapter are separated from the fluid access assembly.

In some embodiments, the scrubbing cap includes a seal attached over the cavity to seal the scrubbing insert within the cavity, with the method further including removing the seal from the scrubbing cap, to enable positioning of the scrubbing cap over the access port of the vascular access device and contacting of the scrubbing insert with the access port.

In some embodiments, cleaning the access port comprises applying a twisting motion to the blood sample collection system.

### BRIEF DESCRIPTION OF THE DRAWINGS

FIG. 1 is a schematic illustration of a blood sample collection system, according to an embodiment;

FIG. 2 is a side view of a blood sample collection system, according to an embodiment;

FIG. 3A is a rear perspective view of the blood sample collection system of FIG. 2 in a first configuration;

FIG. 3B is a rear perspective view of the blood sample collection system of FIG. 2 in a second configuration;

FIG. 4 is an exploded view of a scrubbing cap included in the blood sample collection system of FIG. 2, according to an embodiment;

FIG. 5 is a cross-sectional view illustrating the scrubbing cap of FIG. 4 engaged with an access port of a vascular access device;

FIG. 6 is a side view of a blood sample collection system, according to another embodiment;

FIG. 7 is a side view of a blood sample collection system, according to another embodiment; and

FIG. 8 is a side view of a blood sample collection system, according to another embodiment.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw device in preparation for or during use. Similarly, in this application, the terms “in the distal direction” and “distally” mean in the direction toward an access connector portion of the fluid transfer device, and the terms “in the proximal direction” and “proximally” mean in the direction opposite the direction of the connector.

While not shown or described herein, it is to be understood that the blood sample collection systems described below may be utilized for blood draw from any suitable vascular access device (VAD) such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Embodiments of the present disclosure will primarily be described in the context of blood sample collection systems for use with PIVCs. However, embodiments of the present disclosure equally extend to use with other catheter devices.

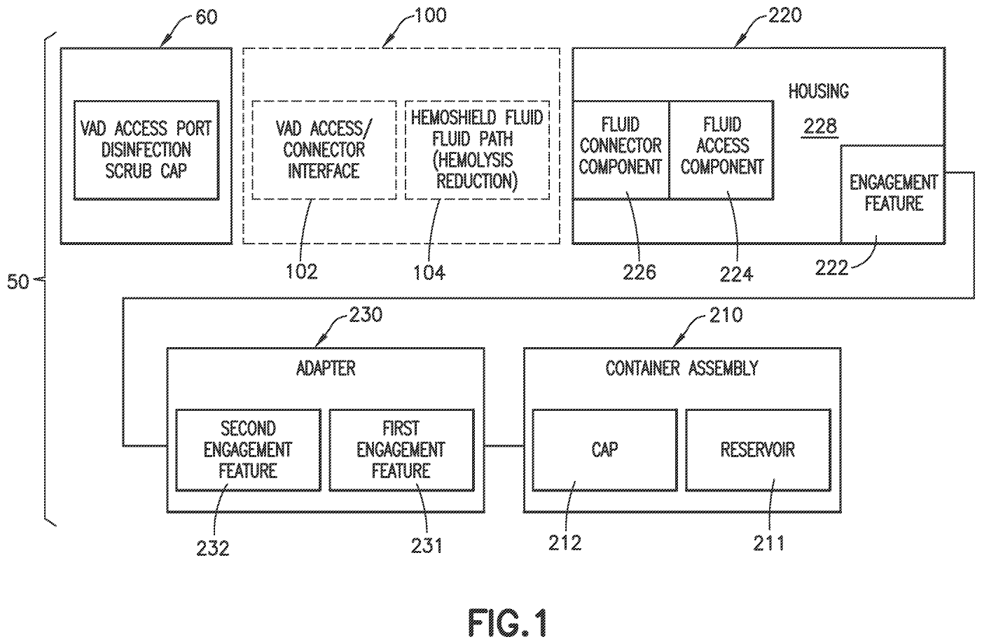

Referring to FIGS. 1-3, a blood sample collection system 50 in accordance with an aspect of the present disclosure is shown. FIG. 1 is a schematic illustration of the blood sample collection system 50 in accordance with one embodiment. The system 50 includes a container assembly 210, an adapter 230, a fluid access assembly 220, and a scrubbing cap 60.

While described in detail below, it is recognized that the container assembly 210 may be any suitable standard evacuated tube and the fluid access assembly 220, can be any suitable standard holder, with the container assembly 210 provided as a BD VACUTAINER® from Becton, Dickinson and Co. and the fluid access assembly 220 provided as a BD VACUTAINER® Holder device, according to one non-limiting example. More generally, the container assembly 210 may include a cap 212 and can define a reservoir 211, while the fluid access assembly 220 may include a housing 228 defining an interior, an engagement feature 222, a fluid access component 224, and a fluid connector component 226.

The adapter 230 may include a first engagement feature 231 and a second engagement feature 232. The first engagement feature 231 of the adapter 230 can be configured to releasably engage with the cap 212 of the container assembly 210, while the second engagement feature 232 of the adapter 230 can be configured to releasably engage with the engagement feature 222 of the fluid access assembly 220. The first engagement feature 231 of the adapter 230 can be or include any suitable engagement mechanism configured to temporarily, or releasably, hold the cap 212 in a position relative to the adapter 230 and to release the cap 212 upon a movement of the cap 212 relative to the first engagement feature 231. The movement can include, for example, a translational movement, a rotational movement, and/or a helical movement. The second engagement feature 232 of the adapter 230 and the engagement feature 222 of the fluid access assembly 220 can be or include any suitable engagement mechanism configured to temporarily, or releasably, engage with each other to temporarily hold the adapter 230 in a position relative to the fluid access assembly 220 or a component of the fluid access assembly 220 (e.g., the housing 228) and to release the adapter 230 from the fluid access assembly 220 (e.g., via a deformation of the adapter 230 resulting from the release of the cap 212 from the adapter 230 and/or via a deformation and/or movement (e.g., rotational, helical, and/or translational) of the second engagement feature 232 and/or the engagement feature 222 relative to the other of the second engagement feature 232 or the engagement feature 222 such that the second engagement feature 232 can be separated from the engagement feature 222).

The container assembly 210 may include a distal end and a proximal end. The cap 212 can be disposed at the distal end of the container assembly 210. In some embodiments, the container assembly 210 may include a tube having an open end and a closed end opposite the open end. The cap 212 can be coupled to the open end such that the cap 212 and the tube define the reservoir 211. In some embodiments, the cap 212 may include a resealable membrane. The resealable membrane may be configured such that a fluid access component, such as fluid access component 224, can pierce the rescalable membrane to achieve fluidic communication with the reservoir 211. The rescalable membrane of the cap 212 may be configured to rescal upon decoupling the fluid access component 224 from the cap 212 such that the reservoir 211 is fluidically isolated from an area external to the container assembly 210. In some embodiments, cap 212 may include ridges and/or one or more flanges disposed on an external surface thereof. The reservoir 211 can be an evacuated reservoir such that, upon the reservoir 211 being placed in fluidic communication with a source of fluid (e.g., via piercing the resealable membrane of the cap 212 with a fluid access component fluidically coupled to a patient's vasculature), fluid (e.g., blood) can be drawn into the reservoir 211 due to a pressure differential between the reservoir 211 and the source of fluid. In some embodiments, the container assembly 210 may be an evacuated tube, while the cap 212 may be formed of any appropriate material such as, e.g., rubber. The tube may be formed of, e.g., plastic.

The fluid access assembly 220 may also have a distal end and a proximal end. The fluid access component 224 may be disposed within the interior of the housing 228 and may extend from the distal end of the housing 228 into the interior of the housing 228. For example, in some embodiments, the fluid access component 224 may have a distal end and a proximal end opposite the distal end. The distal end of the fluid access component 224 may be coupled to the distal end of the housing 228 and the proximal end may be disposed in the interior of the housing 228. In some embodiments, the fluid access component 224 may include a needle defining a lumen. In some embodiments, the fluid access assembly 220 may include a flexible needle sheath configured substantially surround and be translated relative to the needle such that a proximal end of the needle may be selectively exposed.

The fluid connector component 226 may be integrated with or removably coupled to the distal end of the housing 228. For example, the housing 228 may define an outlet fluidically coupled to the lumen of the fluid access component 224 to which the fluid connector component 226 can be coupled. In some embodiments, the fluid connector component 226 may include any suitable component configured to couple the housing 228 to a connector or access port of patient access tubing. For example, in some embodiments, the fluid connector component 226 may be a luer connector. The luer connector may be in the form of, e.g., a slip luer, a threaded luer, a luer lock with collar, etc. In some embodiments, the fluid connector component 226 can be an outlet of the housing 228 defining a lumen.

As shown in FIGS. 1-3, the engagement feature 222 of the fluid access assembly 220 may include a flange extending perpendicularly from a central axis of the housing 228. In some embodiments, the flange can be elongated such that the flange extends farther from the central axis of the housing 228 in a first direction than in a second direction. For example, the flange can form an elongated surface of the fluid access assembly 220 disposed in a plane containing the proximal end of the housing 228.

The first engagement feature 231 of the adapter 230 may be any suitable feature configured to releasably engage with the cap 212 of the container assembly 210. In some embodiments, the first engagement feature 231 of the adapter 230 may be an inner surface of the adapter 230 that defines a through-hole. The inner surface of the adapter 230 may include a diameter sufficiently small relative to an outermost diameter of the cap 212 such that the inner surface and the cap 212 may be engaged via a friction fit. In some embodiments, the first engagement feature 231 can include a feature corresponding to a feature on the cap 212 such that the adapter 230 and the cap 212 can be releasably engaged.

The second engagement feature 232 of the adapter 230 may be any suitable feature configured to releasably engage with the engagement feature 222 of the fluid access assembly 220. For example, the second engagement feature 232 may include two oppositely-disposed tabs. A latch can be disposed on the end of each tab. Each latch may be shaped and sized to receive a portion of a flange of the engagement feature 222 of the fluid access assembly 220. In some embodiments, the adapter 230 may be rotatable relative to the fluid access assembly 220 such that the second engagement feature 232 (e.g., the latches) can be rotated out of engagement with the engagement feature 222 of the fluid access assembly 220 (e.g., a flange). In some embodiments, the second engagement feature 232 can include a number of arms (e.g., two), each of the arms having a distal end coupled to a base of the adapter 230 via a flexible joint and a latch disposed on the opposite end of the arm. In some embodiments, each of the arms may be curved and may form a portion of the outer perimeter of the adaptor 230. Such an adapter can be decoupled from the engagement feature 222 of the fluid access assembly 220 via, for example, rotation and/or deformation (e.g., bending). In some embodiments, the second engagement feature 232 may include a number of tabs including latches configured to be snapped over a flange of the engagement feature 222. For example, the second engagement features 232 may include two or three latching tabs. To separate such an adapter 230 from the housing 228, a user can decouple each latching tab by pulling the latch away from the flange of the engagement feature 222 such that the tab is released from engagement with the flange of the engagement feature 222.

According to embodiments, the adapter 230 allows for container assemblies 210 of various sizes and shapes to be used with (e.g., disposed within and stabilized by) the fluid access assembly 220. As described above, the adapter 230 may be configured (e.g., shaped and sized) to engage with the engagement feature 222 of the fluid access assembly 220, with the second engagement feature 232 of adapter 230 receiving a portion of the engagement feature 222 when the second engagement feature 232 of the adapter 230 is engaged with the engagement feature 222 of the fluid access assembly 220.

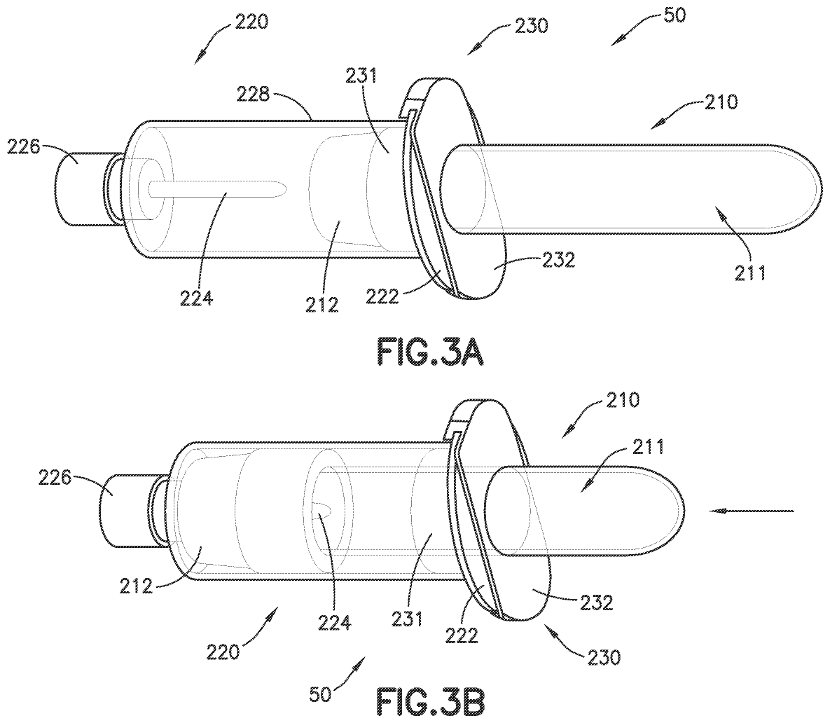

Referring to FIG. 3A, the system 50 has a first configuration (e.g., an initial configuration) in which the first engagement feature 231 of the adapter 230 is engaged with the cap 212 and the second engagement feature 232 is engaged with the engagement feature 222 of the fluid access assembly 220. When the first engagement feature 231 of the adapter 230 is engaged with the cap 212 and the second engagement feature 232 is engaged with the engagement feature 222 of the fluid access assembly 220, the cap 212 is spaced away from the fluid access component 224 such that the reservoir 211 is fluidically isolated from an external environment of the container assembly 210. In the first configuration, the cap 212 may be at least partially disposed within the adapter 230 and/or the housing 228. For example, a distal end of the cap 212 may be disposed within the interior of the housing 228 (either projecting from the adapter 230 or within the adapter 230). In some embodiments, the distal end of the cap 212 may be disposed and retained within the adapter 230 but proximal of the housing 228 in the first configuration. In some embodiments, the entire system 50 can be sterilized in the first configuration and packaged for sterile transport to a user (e.g., a healthcare provider).

Next, referring to FIG. 3B, the system 50 also has a second configuration in which the lumen of the fluid access component 224 is in fluidic communication with the reservoir 211. To transition the system 50 from the first configuration to the second configuration, the container assembly 210 may be distally translated toward the distal end of the fluid access assembly 220 such that the cap 212 is engaged with the fluid access component 224 (e.g., the fluid access component 224 pierces the cap 212) and a portion of the fluid access component 224 is disposed within the reservoir 211. For example, in embodiments in which the container assembly 210 is engaged with the first engagement feature 231 via a friction fit, a force may be applied to the container assembly 210 to overcome the force applied by the first engagement feature 231 on the container assembly 210 (e.g., the cap 212) and translate the container assembly 210 into engagement with the fluid access component 224. In the second configuration, when the fluid connector component 226 is fluidically coupled to a patient's vasculature system (via connection of blood sample collection system 50 to a VAD, such as via coupling of fluid connector component 226 to a VAD access port), fluid (e.g., blood) can be drawn through the fluid connector component 226, through the fluid access component 224, and into the reservoir 211 of the container assembly 210.

When sufficient blood has been drawn into the reservoir 211, the system 50 may be transitioned from the second configuration to a third configuration (not shown) in which the container assembly 210 and the adapter 230 are separated from the fluid access assembly 220. For example, the container assembly 210 may be translated relative to the fluid access assembly 220 such that the cap 212 is disposed near the proximal end of the housing 228. In response to the cap 212 being disengaged from the fluid access component 224, the reservoir 211 may be fluidically isolated from an environment external to the container assembly 210 due to the cap 212 having a rescalable membrane. The adapter 230 can then be decoupled from the fluid access assembly 220 via decoupling the second engagement feature 232 from the engagement feature 222 via, e.g., rotating, unsnapping, or deforming one or more portions of the second engagement feature 232 from the engagement feature 222 of the fluid access assembly 220. The adapter 230 and the container assembly 210 may then be optionally discarded, particularly as this initial blood sample may be considered the “discard sample”.

After removing the adapter 230 and the container assembly 210 from the fluid access assembly 220, a second container assembly may be inserted into the interior of the housing and engaged with the fluid access component 224 such that the second container assembly can draw blood into a reservoir of the second container assembly via the fluid connector component 226 and the fluid access component 224. In some embodiments, the second container assembly may include a medium (e.g., a soybean casein digest broth) in the reservoir of the second container configured to be used to perform a blood culture when combined with the patient's blood sample in the reservoir. Any suitable number of container assemblies can be engaged (and subsequently disengaged) with the fluid access component 224 to draw fluid from a patient for various tests. Furthermore, because many of the primary components of system 50 do not need to be detached between blood draws, workflow for the healthcare provider is improved. Additionally, the overall reduction in connections made as compared to conventional blood draw methods may reduce the risk of sample contamination during blood draw. This, coupled with the ability to provide the entirety of system 50 in sterile packaging, reduces the overall risk of contamination of the blood culture samples.

According to aspects of the disclosure, it is recognized that when initially connecting the blood sample collection system 50 to a VAD access port, such as via coupling of the fluid connector component 226 with the access port, it is desirable to disinfect the access port prior to connection of blood sample collection system 50 thereto. That is, it is desirable to clean and sterilize the access port prior to engagement of fluid connector component 226 therewith (e.g., via mating of female and male luer connections) to prevent microbial ingress and possible catheter-related blood stream infections (CRBSIs).

According to aspects of the disclosure, a scrubbing cap 60 is integrated in with the blood sample collection system 50 at the distal end 52 thereof to provide for cleaning and sterilizing of the access port prior to engagement of the system 50 therewith. The scrubbing cap 60 is secured onto fluid connector component 226 and is configured to engage with an access port to enable the cleaning and sterilizing of internal and/or external surfaces thereof, such as via a twisting and scrub motion between the scrubbing cap 60 and access port. The scrubbing cap 60 may be removed from fluid connector component 226 upon completion of the cleaning and sterilizing of the access port, thereby enabling a subsequent coupling of fluid connector component 226 to the access port and performing of a blood draw via operation of system 50 as previously described.

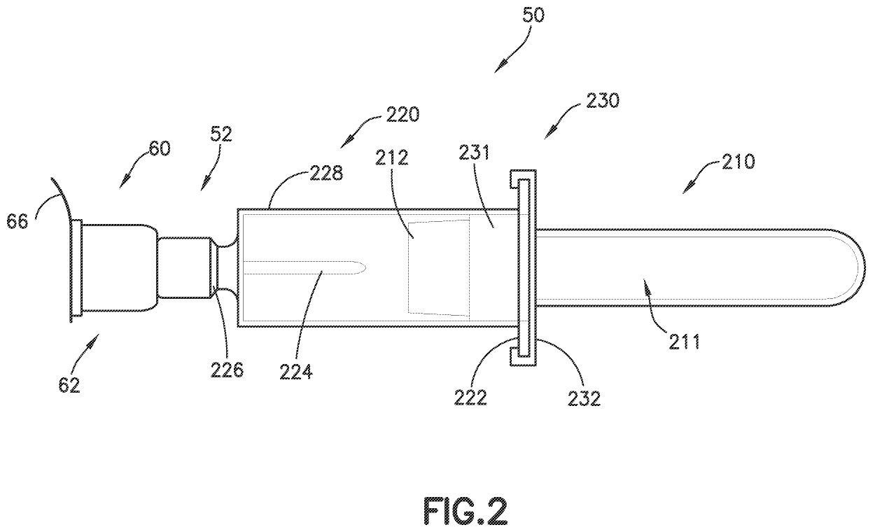

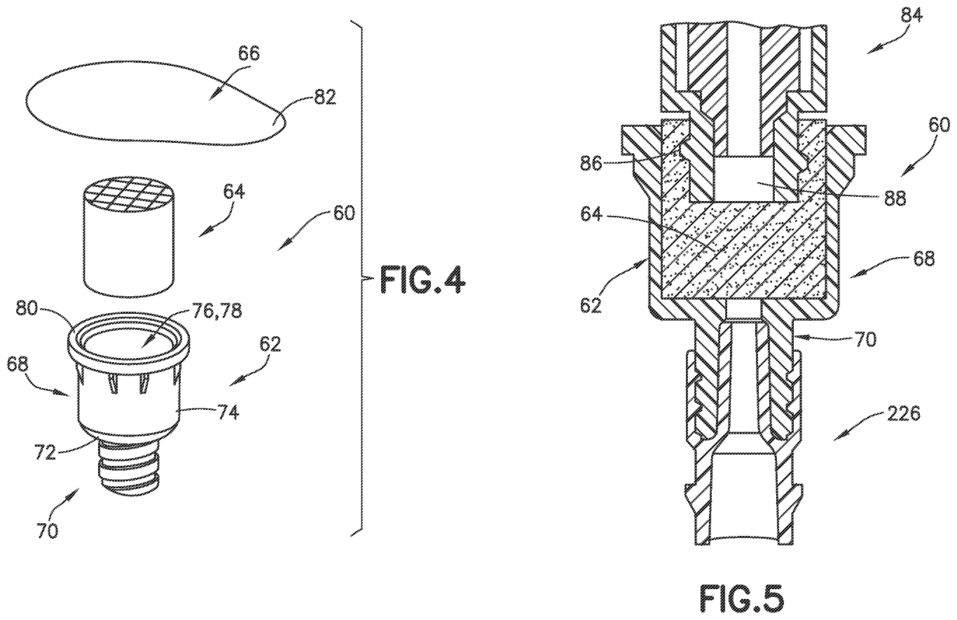

As shown in FIGS. 1 and 2 and now also in FIGS. 4 and 5, scrubbing cap 60 includes a cap housing 62, scrubbing insert 64, and a pealable seal 66. The housing 62 may be an integrally formed structure made by injection molding and may be made of an alcohol compatible material, such as polypropylene or polyethylene, for instance. The housing 62 may generally be described as including a holder portion 68 that retains the insert 64 therein and a connector portion 70 configured to mate with the fluid connector component 226 of blood sample collection system 50.

The holder portion 68 may have a cup shape that is formed by a base 72 and a sidewall structure 74 that defines an opening 76 on one end thereof. The sidewall structure 74 is formed integrally with the base 72 to define a cavity 78 configured to receive the insert 64 therein. In some embodiments, the cavity 78 may have a cylindrical cross-sectional shape and may have a depth suitable to receive the insert 64 therein in a recessed manner. However, it is appreciated that the holder portion 68 and the cavity 78 defined thereby can assume other shapes, including square, hexagonal, etc., and that the holder portion 68, its cavity 78, and the insert 64 disposed therein can be configured in shape and size so as to enable the scrubbing cap 60 to cleanse a particular size and configuration of a medical device with which the scrubbing cap 60 is to be employed (i.e., catheter connector 20 of catheter assembly 12).

The holder portion 68 may be sized such that the cavity 78 defined by sidewall structure 74 compresses the insert 64 when the insert 64 is fitted into the cavity 78, so as to retain the insert 64 therein. A suitable hot melt glue or other suitable adhesive may also be used to adhere the insert 64 to the bottom of the holder portion 68 (i.e., to base 72), although it is recognized that other suitable methods can also be employed to secure the insert 64 to the holder portion 68, including mechanical fixation for instance. An annular lip 80 may be formed on the holder portion 68 (i.e., on sidewall structure 74) about opening 76 to define a land for receiving the seal 66, with the seal 66 cooperating with the annular lip 80 to seal the opening 76 of the cavity 78 and retain the insert 64 therein. The seal 66 seals the cavity 78 of the holder portion 68 and the insert 64 therein against contamination from the outside environment and provides a leak-proof barrier, thereby protecting the contents of insert 64 and maintaining a sealed, sterilized environment. The seal 66 provides a sufficient seal at a range of temperatures, pressures, and humidity levels and, according to embodiments, may be formed as an aluminum or multi-layer polymer film peel back top. In some embodiments, the seal 66 is heat-sealed or induction sealed to the open end of the scrubbing cap 60. The seal 66 can include a tab 82 to facilitate the manipulation of and removal of the seal 66 from the scrubbing cap 60.

The insert 64 is constructed of a foam material, for example, of injection molded construction or the insert 64 may be die-cut from a foam sheet. The insert includes a cleansing substance impregnated therein (while in the holder portion 68), such as a solution of a suitable microbiocide or germicide. The cleansing substance can include an anti-bacterial disinfectant of any suitable type and suitable amount depending upon the size of the insert of foam material. For example, in one embodiment use is made of an aqueous solution including about two percent (2%) chlorhexidine gluconate (chlorhexidine solution, “CHG”) by volume in an amount of from about 0.20 cc to about 0.75 cc. Optionally, a solution including about 0.50 cc is employed. In another embodiment, a solution including about 70 percent (70%) isopropyl alcohol (“IPA”) in an aqueous solution is included in the cleansing substance. In yet another embodiment, a solution including about 70 percent (70%) IPA and about two percent (2%) CHG in an aqueous solution in an amount of about 0.2 ml is included in the cleansing substance. In the latter solution, it is recognized that the concentration of IPA can vary from about 60 percent (60%) to about 90 percent (90%) and the concentration of CHG can vary from about one percent (1%) to about five percent (5%), in one embodiment. Other suitable solution compositions and concentrations are also possible. For instance, povidone iodine or hydrogen peroxide solutions can be included in the cleansing substance, in one embodiment.

According to aspects of the disclosure, the insert 64 may be constructed (e.g., molded) to have a predefined shape that conforms to the unique shape of the VAD access port or connector that is to be cleaned thereby. The insert 64 may further have any of a number of constructions that provide for effective cleaning of the port/connector, such as including a patterned or roughened top surface and/or gaps or slits formed in the foam material thereof that enables the insert 64 to better deform/conform about interior and exterior surfaces of the port/connector.

The connector portion 70 of scrubbing cap 60 extends proximally away from holder portion 68 and is configured to mate with fluid connector component 226 to secure the scrubbing cap 60 thereto. According to embodiments, the connector portion 70 may be configured as a luer connection (e.g., male luer connection or female luer connection) that enables mating thereof with the corresponding luer connection provide by fluid connector component 226, such that the connector portion 70 is secured in place relative to fluid connector component 226.

Following here below, and as shown in FIG. 5, use of the scrubbing cap 60 as a scrubbing device for cleaning and sterilizing of an access port or connector 84 of a VAD is described in accordance with one aspect of the disclosure. Use of the scrubbing cap 60 begins by removing the seal 66 from the scrubbing cap 60. After removal of the seal 66, a connector 84 (which may be configured as a female luer connection having a threaded outer connection 86 defining a tapered lumen or cavity 88, according to a non-limiting embodiment) is inserted by the user into the foamed insert 64. At this time, portions of the insert 64 conform about and/or are inserted into the connector 84 (i.e., about threaded outer connection 86 and into lumen 88). Once the portion of the connector 84 has been inserted into the foamed insert 64 of the scrubbing cap 60, the scrubbing cap 60 is rotated relative to the connector 84. For example, the user may hold the connector 84 stationary while rotating the blood sample collection system 50. As the scrubbing cap 60 is secured in place relative to the blood sample collection system 50—via the connection of fluid connector component 226 to scrubbing cap 60—rotation of the blood sample collection system 50 causes a corresponding rotation of scrubbing cap 60. Rotation of the scrubbing cap 60 relative to the connector 84 may be done all in one direction or may be a back-and-forth twisting motion. The scrubbing cap 60 is rotated a sufficient number of times relative to the connector 84 to sufficiently kill any bacteria that the solution-impregnated foam insert 64 comes in contact with and/or to remove any biofilm from the outside peripheral surface and threaded outer connection 86 of connector 84 as well as the inside surface of the lumen 88 of the connector 84. In this way, both exterior surfaces and interior luminal surfaces of the connector 84 are scrubbed by the insert 64, causing the cleansing substance therein to disinfect the surfaces and remove any biofilm disposed thereon.

Upon completion of the cleaning and sterilizing of the connector 84 of connector 84 via scrubbing cap 60, the connector 84 is removed from the scrubbing cap 60. The scrubbing cap 60 may then be removed from fluid connector component 226 of system 50. After allowing the connector 84 to dry, the blood sample collection system 50 may then be connected thereto by coupling the fluid connector component 226 to connector 84.

While blood sample collection system 50 is shown and described in FIGS. 1-3 according to a specific embodiment, it is recognized that the system 50 may have other suitable configurations. Described here below are a number of alternative constructions for system 50, according to additional aspects of the disclosure.

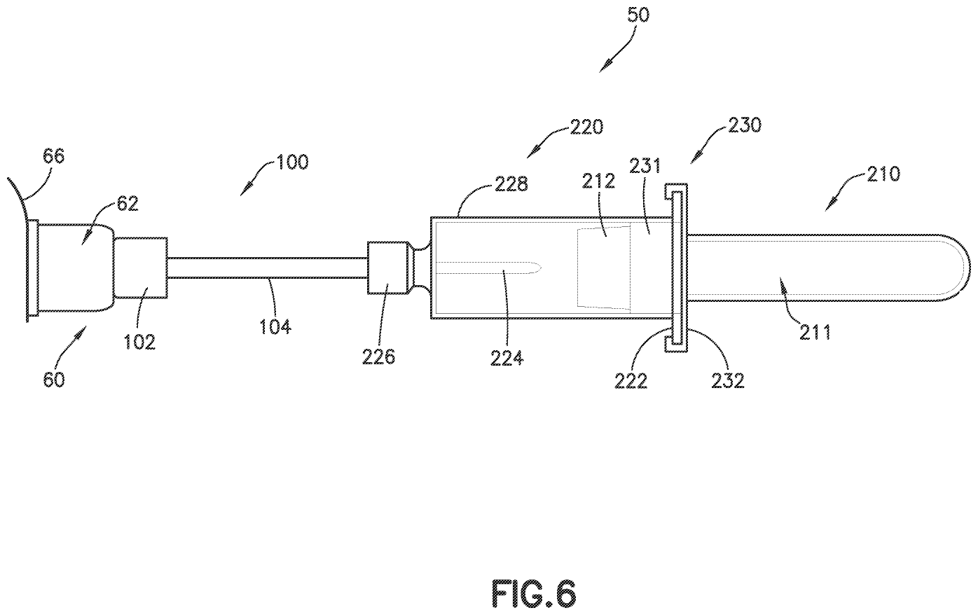

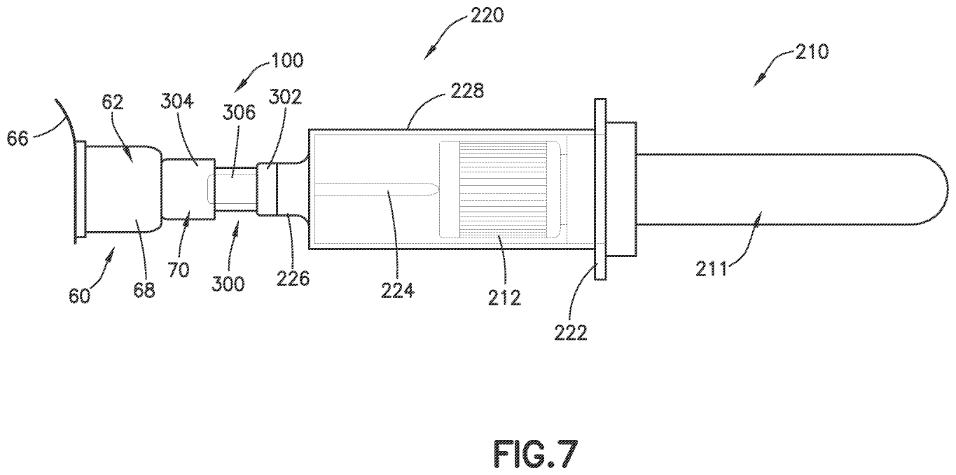

Referring again to FIG. 1 (to the elements shown in phantom therein) and now also to FIGS. 6 and 7, the system 50 is shown as optionally including an extension member 100, wherein extension member 100 is configured to reduce hemolysis of the blood samples drawn into the container assembly 210. In the embodiment of FIG. 6, the extension member 100 may include a connector interface 102 and a fluid resistance-optimized fluid path member 104. The connector interface 102 may be configured as any appropriate interface capable of coupling the extension member 100 to an access port of a VAD. For example, in some embodiments, the connector interface 102 may be configured as a threaded luer, a slip luer, a threaded luer lock with collar, a blunt plastic cannula (with or without alligator-style connection clips), a male luer (with or without alligator-style connection clips), a cannula for PRN access, a needle-free connector, or a needle access cannula.

The fluid path member 104 may be formed of any appropriate element capable of providing optimized fluid resistance so as to reduce hemolysis of the blood sample(s) as the blood flows between the connector interface 102 and the fluid connector component 226. In the embodiment shown in FIG. 6, the fluid path member 104 is formed of flexible tubing. The inner diameter and length of the flexible tubing may be selected so as to limit a maximum blood collection rate which, in turn, may limit a maximum shear stress experienced by the blood cells during blood collection. As noted above, it is shear stress on the blood cells (and particularly on the walls of the blood cells) that is considered a major source of hemolysis and mechanical damage to blood cells. Accordingly, by optimizing fluid path member 104 for fluid resistance, hemolysis of the collected blood sample may be reduced.

However, while FIG. 6 shows the extension member 100 as including a connector interface 102 and a fluid path member 104 formed as flexible tubing, the present disclosure is not limited as such. For example, in some embodiments, extension member 100 may be formed as a compact connector. Referring to FIG. 7, a compact connector 300 in accordance with one aspect of the present disclosure is shown. It is to be understood that compact connector 300 may be utilized in lieu of connector interface 102 and fluid path member 104. The compact connector 300 may include a proximal connector portion 302 configured to couple the compact connector 300 to, e.g., the fluid connector component 226 of fluid access assembly 220. A distal connector portion 304 may be provided for coupling the compact connector 300 to, e.g., an access port of a vascular access device. Additionally, a central portion 306 may be provided, with the central portion 306 shaped and configured to increase flow resistance and, thus, reduce hemolysis. Other configurations for fluid path member 104 are also possible. For example, flow path 104 may be configured similar to one or more fluidic resistance-optimized flow paths described in any one of U.S. application Ser. No. 17/146,388, U.S. application Ser. No. 17/401,506, and U.S. application Ser. No. 17/496,858, the disclosures of which are incorporated herein by reference in their entirety.

In the embodiments of FIGS. 6 and 7, scrubbing cap 60 (as shown in detail in FIG. 4) is provided on the distal end of extension member 100, such as on connector interface 102 (or distal connector portion 304, if the extension member is provided as a compact connector 300). The scrubbing cap 60 is configured as described in detail above, but the connector portion 70 of scrubbing cap 60 mates with connector interface 102 to secure the scrubbing cap 60 thereto. As indicated above, the connector interface 102 may be configured as a threaded luer, a slip luer, a threaded luer lock with collar, a blunt plastic cannula (with or without alligator-style connection clips), a male luer (with or without alligator-style connection clips), a cannula for PRN access, a needle-free connector, or a needle access cannula, and the connector portion 70 may be configured to have a corresponding construction that provides for mating of the scrubbing cap 60 with the connector interface 102, with one embodiment having the connector interface 102 structure as a female luer connection, for example.

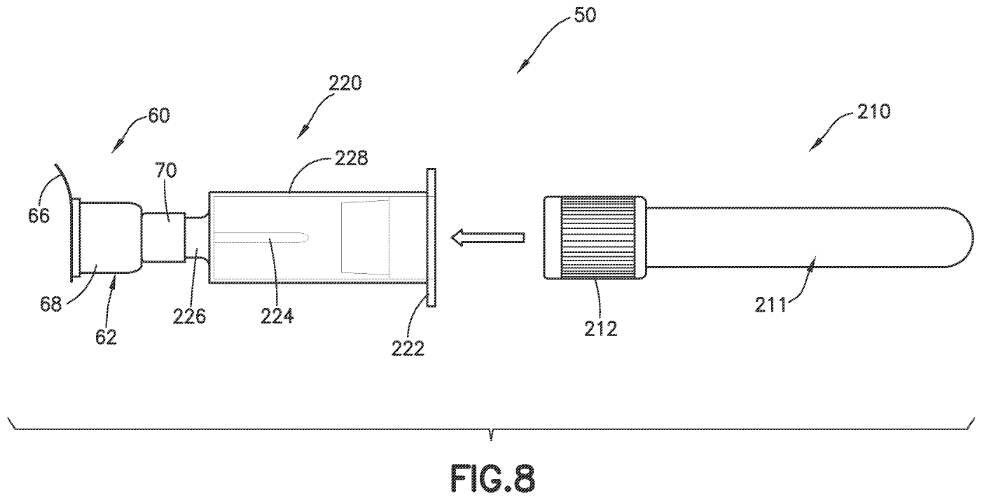

Referring now to FIG. 8, the blood sample collection system 50 is shown according to still another embodiment, where the system 50 does not include either of the extension member 100 or the adapter 230—i.e., the system 50 includes only container assembly 210, fluid access assembly 220, and scrubbing cap 60. In the illustrated embodiment, scrubbing cap 60 engages/mates with fluid access assembly 220 in the same manner as done in the system of FIGS. 1-5—with the connector portion 70 of scrubbing cap 60 configured to mate with fluid connector component 226 to secure the scrubbing cap 60 thereto. As previously described, in some embodiments, the connector portion 70 may be configured as a luer connection (e.g., male luer connection or female luer connection) that enables mating thereof with the corresponding luer connection provide by fluid connector component 226, such that the connector portion 70 is secured in place relative to fluid connector component 226.

While embodiments of system 50 described above with respect to FIGS. 1-8 utilize fluid access assembly 220, adapter 230, container assembly 210 and extension member 100 in conjunction with the scrubbing cap 60, it is to be understood that the present disclosure is not limited as such. That is, the scrubbing cap 60 may be utilized with other blood collection assemblies, including those shown and described in various embodiments of U.S. Application Publication No. 2021/0196167, which is incorporated herein by reference in its entirety.

While several embodiments of blood sample collection system configured for blood draw during catheter indwell were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is:

1. A system, comprising:
a container assembly comprising a cap and defining a reservoir, the container assembly having a distal end and a proximal end, the cap disposed at the distal end of the container assembly;
a fluid access assembly comprising a housing defining an interior, a fluid access component, and a fluid connector component, the housing having a distal end and a proximal end, the fluid access component extending from the distal end of the housing into the interior of the housing, the fluid access component defining a lumen, the fluid connector component disposed on the distal end of the housing; and
a scrubbing cap provided at or adjacent a distal end of the fluid access assembly, the scrubbing cap comprising:
a scrubbing insert; and
a housing defining a cavity configured to retain the scrubbing insert therein.

2. The system of claim 1, wherein the fluid connector component comprises a luer connector configured to be coupled to an access port of a vascular access device, and wherein the scrubbing cap comprises a connector portion configured to engage with the luer connector to secure the scrubbing cap to the fluid access assembly.

3. The system of claim 2, wherein the luer connector comprises one of a slip luer, a threaded luer, or a luer lock with collar.

4. The system of claim 1, comprising an adapter including a first engagement feature and a second engagement feature, the first engagement feature of the adapter configured to releasably engage with the cap of the container assembly and the second engagement feature of the adapter configured to releasably engage with a housing engagement feature of the fluid access assembly such that, in a first configuration in which the first engagement feature of the adapter is engaged with the cap and the second engagement feature of the adapter is engaged with the engagement feature of the fluid access assembly, the cap of the container assembly is at least partially disposed within the interior of the housing and spaced from the fluid access component.

5. The system of claim 4, wherein the container assembly is configured to be transitioned from the first configuration to a second configuration via translating the container assembly toward the first end of the fluid access assembly such that the cap is disengaged from the first engagement feature of the adapter and the fluid access component pierces a resealable membrane of the cap such that the reservoir of the container assembly is in fluidic communication with the fluid connector component and the connection portion via the lumen of the fluid access component.

6. The system of claim 1, comprising an extension member having a distal end and a proximal end, the proximal end of the extension member coupled to the fluid connector component, wherein the extension member is configured to reduce hemolysis of a blood sample passing therethrough, and wherein the scrubbing cap comprises a connector portion configured to engage with the distal end of the extension member to secure the scrubbing cap to the extension member.

7. The system of claim 6 wherein the extension member comprises:
a connector interface disposed at the distal end and configured to couple the extension member to an access port of a vascular access device; and
a flexible tube fluidly coupled to the connector interface and configured to be coupled to the fluid connector component of the fluid access assembly, wherein the flexible tube is configured to reduce hemolysis of a blood sample passing therethrough.

8. The system of claim 6, wherein the extension member comprises a compact connector, the compact connector comprising:
a proximal connector portion configured to couple the compact connector to the fluid connector component;
a distal connector portion configured to couple the compact connector to an access port of a vascular access device; and
a central portion shaped and configured to reduce hemolysis of a blood sample passing therethrough.

9. The system of claim 7, wherein the connector interface or the distal connector comprises one of a threaded luer, a slip luer, a threaded luer lock with collar, a blunt plastic cannula, a male luer, a cannula for PRN access, a needle-free connector, or a needle access cannula.

10. The system of claim 2, wherein the connector portion of the scrubbing cap secures the scrubbing cap via a twist-type engagement, and wherein the scrubbing cap is removable to enable connection of the system to an access port of a vascular access device.

11. The system of claim 1, wherein the scrubbing insert comprises a resilient material including an antimicrobial solution or agent absorbed therein configured to disinfect a surface of the access port of the vascular access device.

12. The system of claim 1, wherein the scrubbing cap comprises a seal attached over the cavity to seal the scrubbing insert within the cavity.

13. A method of using a blood sample collection system, comprising:
providing the blood sample collection system, the system comprising:
a container assembly comprising a cap and defining a reservoir, the container assembly having a distal end and a proximal end, the cap disposed at the distal end of the container assembly,
a fluid access assembly comprising a housing defining an interior, a fluid access component, and a fluid connector component, the housing having a distal end and a proximal end, the fluid access component extending from the distal end of the housing into the interior of the housing, the fluid access component defining a lumen, the fluid connector component disposed on the distal end of the housing, and
a scrubbing cap provided on or adjacent the distal end of the housing, the scrubbing cap comprising:
a scrubbing insert; and
a housing defining a cavity configured to retain the scrubbing insert therein;
positioning the scrubbing cap over an access port of a vascular access device, such that the access port contacts the scrubbing insert; and
cleaning the access port with the scrubbing insert.

14. The method of claim 13, wherein the fluid connector component comprises a luer connector configured to be coupled to the access port, and wherein the scrubbing cap comprises a connector portion engaged with the luer connector to secure the scrubbing cap to the fluid access assembly.

15. The method of claim 14, comprising:
detaching the scrubbing cap from the luer connector subsequent to cleaning of the access port; and
coupling the luer connector to the access port, to place the fluid access assembly in fluid connection with the vascular access device.

16. The method of claim 13, wherein the system comprises an extension member having a distal end and a proximal end, the extension member configured to reduce hemolysis of a blood sample passing therethrough, and wherein the scrubbing cap comprises a connector portion engaged with the distal end of the extension member to secure the scrubbing cap to the extension member.

17. The method of claim 16, comprising:
detaching the scrubbing cap from the distal end of the extension member subsequent to cleaning of the access port; and
coupling the distal end of the extension member to the access port, to place the fluid access assembly in fluid connection with the vascular access device.

18. The method of claim 13, wherein the system comprises an adapter including a first engagement feature and a second engagement feature, the first engagement feature of the adapter configured to releasably engage with the cap of the container assembly and the second engagement feature of the adapter configured to releasably engage with a housing engagement feature of the fluid access assembly such that, in a first configuration in which the first engagement feature of the adapter is engaged with the cap and the second engagement feature of the adapter is engaged with the engagement feature of the fluid access assembly, the cap of the container assembly is at least partially disposed within the interior of the housing and spaced from the fluid access component.

19. The method of claim 18, comprising:
translating the container assembly toward the first end of the fluid access assembly and relative to the adapter such that the cap is disengaged from the first engagement feature of the adapter and the fluid access component pierces a resealable membrane of the cap such that the reservoir of the container assembly is in fluidic communication with the fluid connector component via the lumen of the fluid access component;
decoupling the second engagement feature of the adapter from the engagement feature of the fluid access assembly; and
translating the container assembly away from the first end of the fluid access assembly and out of the interior of the fluid access assembly such that the container assembly and the adapter are separated from the fluid access assembly.

20. The method of claim 13, wherein the scrubbing cap comprises a seal attached over the cavity to seal the scrubbing insert within the cavity, and wherein the method further comprises removing the seal from the scrubbing cap, to enable positioning of the scrubbing cap over the access port of the vascular access device and contacting of the scrubbing insert with the access port.
